(19) United States
(12) Patent Application Publication
(10) Pub. No.: US 2025/0339664 A1
(43) Pub. Date: Nov. 6, 2025

(54) CIRCULATORY ASSIST DEVICE WITH PULSATILE STENT GRAFT INTEGRATED INTO STENT CAGE

(71) Applicant: PULSEGRAFT, INC., Corona Del Mar, CA (US)

(72) Inventors: Howard J. Leonhardt, Mission Viejo, CA (US); Jeff Donofrio, Anaheim, CA (US)

(21) Appl. No.: 18/870,265

(22) PCT Filed: May 30, 2023
(86) PCT No.: PCT/US2023/023892

§ 371 Date: Nov. 27, 2024

Related U.S. Application Data
(60) Provisional application No. 63/348,469, filed on Jun. 2, 2022.

(51) Int. Cl. A61M 60/135; A61M 60/216; A61M 60/422; A61M 60/562; A61M 60/806

(52) U.S. Cl. CPC A61M 60/135; A61M 60/216; A61M 60/422; A61M 60/562; A61M 60/806

(57) ABSTRACT

A circulatory assist device for facilitating pulsatile blood flow within a subject's blood vessel is disclosed. The circulatory assist device includes a rotary component, a pulsatory component, and one or more electromagnets. The rotary component includes a driveline and one or more impellers connected to the driveline and configured to rotate with the driveline. The pulsatory component includes one or more sections configured to change diameter in response to a magnetic field applied thereto. The one or more electromagnets are positioned within the driveline and are configured to produce the magnetic field

### CROSS-REFERENCE TO RELATED APPLICATIONS

This application is a national phase entry of International Patent Application PCT/US2023/023892, filed May 30, 2023, designating the United Kingdom and published in English as International Patent Publication WO2023/235330 A1 on Dec. 7, 2023, which claims the benefit under Article 8 of the Patent Cooperation Treaty to U.S. Provisional Patent Application Ser. No. 63/348,469, filed Jun. 2, 2022.

### TECHNICAL FIELD

The application relates generally to medical devices, and more particularly to an apparatus, system, and associated methods for assisting a subject's heart to pump blood.

### BACKGROUND

There are a variety of types of circulatory assist devices that facilitate blood flow within the subject's (e.g., mammal, such as a human) blood vessels.

External vessel circulatory assist devices may cause damage to blood vessels, have been inconsistent in performance, and need to be placed surgically with relatively invasive procedures. Some circulatory assist devices have been known to migrate out of position, or require suture sewing, hooks, and/or barbs to be held in place. Additionally, many circulatory assist devices are too rigid for the affected artery to maintain contact with the vessel wall, while also allowing for vessel wall pulsaltility.

Many internal devices have been known to cause damage to blood cells and have a relatively high risk of leading to blood clotting and most often also eliminate arterial vessel wall or blood flow pulsaltility.

U.S. Pat. No. 8,617,239 to Reitan (Dec. 13, 2013), U.S. Pat. No. 8,617,239 to Reitan, which builds upon an earlier patent of Reitan, i.e., U.S. Pat. No. 5,749,855 to Reitan (May 12, 1998), and U.S. Patent Application Publication 2022/0117719 A1 to Leonhardt (Apr. 21, 2022) for “Pulsatile Vascular Stent Graft,” the contents of each of which are hereby incorporated by this reference, each describe devices that utilize an impeller to facilitate blood flow within the subject's blood vessels.

In addition, some current stent graft devices utilize electrical current for actuation, such as that described in Palma et al., “Pulsatile stent graft: a new alternative in chronic ventricular assistance,” Revista Brasileira de Cirurgia Cardiovascular (2013), 28(2):217; dx.doi.org/10.5935/1678-9741.20130031, the contents of which are incorporated herein by this reference, which may not be desirable in certain circumstances.

U.S. Patent Application Publication 2022/0117719 A1 to Leonhardt (Apr. 21, 2022) for “Pulsatile Vascular Stent Graft,” the contents of which are incorporated herein by this reference, describes an intravascular device that includes a stent structure and at least one annular band that is configured to be electronically activated. In some embodiments, the annular band may be an electro-activated polymer, such as a ferroelectric polymer or a dielectric polymer. Accordingly, an electrical (or, e.g., magnetic) field may be applied to the annular band and the annular band will change shape (e.g., expand and/or contract) in response to the applied electric field such as a voltage.

The above-described background relating to circulatory assist devices is merely intended to provide a contextual overview of some current issues and is not intended to be exhaustive. Other contextual information may become apparent to those of ordinary skill in the art upon review of the following description, which includes example embodiments.

### BRIEF SUMMARY

A circulatory assist device generally includes a rotary component and a pulsatory component. The rotary component includes an impeller encompassed by at least a portion of a stent cage. The pulsatory component includes a stent graft integrated in the at least a portion of the stent cage. At least a portion of the stent graft is configured to change diameter in response to an applied stimulus. The impeller and the stent graft act cooperatively to facilitate blood flow within the blood vessel of the subject. Methods of facilitating pulsatile blood flow in a blood vessel of a subject are also described.

Particularly described is a circulatory assist device comprising: a stent cage, a pulsatory component configured to facilitate pulsatile blood flow through a blood vessel of a subject, the pulsatory component comprising a stent graft integrated in at least a portion of the stent cage, the stent graft including one or more sections configured to diametrically constrict in response to an applied stimulus, and a rotary component configured to facilitate the pulsatile blood flow through the blood vessel of the subject, the rotary component comprising an impeller encompassed by at least a portion of the stent graft, the impeller and the one or more sections are configured to act cooperatively to facilitate blood flow within the blood vessel of the subject.

In certain embodiments, the circulatory assist device has each of the one or more sections includes a ferroic material configured to cause a respective section of the one or more sections to constrict in response to the applied stimulus.

In certain embodiments of the circulatory assist device, the impeller includes a driveline and one or more impeller blades extending from the driveline.

In certain embodiments, the circulatory assist device further comprises at least one electromagnet positioned within the driveline, and wherein the electromagnet(s) are configured to cause the applied stimulus.

In certain embodiments, the circulatory assist device further comprises circuitry configured to cause constriction and expansion of the one or more sections by controlling the electromagnet(s) and emission of the applied stimulus thereby.

In certain embodiments, the circulatory assist device further comprises a motor coupled to the driveline, and wherein the circuitry is configured to control rotation of the impeller via the motor simultaneously with the constriction and the contraction of the one or more sections.

In certain embodiments of the circulatory assist device, the each of the stent cage, the pulsatory component, and the rotary component is configured to transition from a stowed position including a radial footprint substantially small enough to fit within an outer casing of a catheter and a deployed position including a radially expanded footprint substantially large enough for the stent cage to contact an inner wall of the blood vessel.

Also described is a circulatory assist device for facilitating pulsatile blood flow within a subject's blood vessel, the circulatory assist device comprising: a rotary component comprising a driveline and one or more impellers connected to the driveline and configured to rotate with the driveline; a pulsatory component comprising one or more sections configured to change diameter in response to a magnetic field applied thereto; and one or more electromagnets positioned within the driveline and configured to produce the magnetic field.

In certain embodiments of the circulatory assist device, each of the one or more sections includes a ferroic material configured to constrict in response to the magnetic field being applied thereto.

In certain embodiments of the circulatory assist device, each of the pulsatory component and the rotary component is configured to transition from a stowed position including a radial footprint substantially small enough to fit within an outer casing of a catheter and a deployed position including a radially expanded footprint larger than the radial footprint.

In certain embodiments, the circulatory assist device further comprises circuitry configured to cause constriction and expansion of the one or more sections by controlling the one or more electromagnets and production of the magnetic field thereby.

In certain embodiments, the circulatory assist device further comprises a motor coupled to the driveline, and wherein the circuitry is configured to control rotation of the impeller via the motor simultaneously with the constriction and the contraction of the one or more sections.

In certain embodiments, the circulatory assist device further comprises a stent cage positioned on each side of the pulsatory component, the stent cage on each side of the pulsatory component configured to support the pulsatory component.

In certain embodiments of the circulatory assist device, the ferroic material includes one or more material selected from among ferroelectric material and ferromagnetic material.

Methods of making and using the circulatory assist devices are also described. In use, such a method includes facilitating pulsatile blood flow within a blood vessel of a subject (e.g., a mammalian subject, such as a human in need thereof), the method comprising: introducing a circulatory assist device into the blood vessel, the circulatory assist device including a rotary component and a pulsatory component; causing a driveline of the rotary component to rotate one or more impellers connected to thereto; and causing one or more sections of the pulsatory component to change diameter by applying a magnetic field thereto using one or more electromagnets positioned within the driveline, the one or more electromagnets configured to produce the magnetic field.

In certain embodiments, the method further comprises causing each of the pulsatory component and the rotary component to transition from a stowed position including a radial footprint substantially small enough to fit within an outer casing of a catheter to a deployed position including a radially expanded footprint larger than the radial footprint after introducing the circulatory assist device into the blood vessel.

In certain embodiments of the method, the circulatory assist device includes a stent cage positioned at least on each side of the pulsatory component and the radially expanded footprint is substantially large enough for the stent cage to contact an inner wall of the blood vessel.

In certain embodiments, the method further comprises, prior to removing the circulatory assist device from the blood vessel causing each of the pulsatory component and the rotary component to transition to the stowed position from the deployed position including causing one or more impeller blades of the impeller to be stowed within pockets formed by a casing of the driveline.

In certain embodiments of the method, causing the driveline of the rotary component to rotate the one or more impellers connected to thereto and causing the one or more sections of the pulsatory component to change diameter by applying the magnetic field thereto are performed simultaneously.

In certain embodiments of the method, the one or more sections include a ferroic material configured constrict in response to the magnetic field being applied thereto.

### BRIEF DESCRIPTION OF THE DRAWINGS

FIG. 1 shows a circulatory assist device in a stowed (e.g., collapsed state) position, in accordance with embodiments of this disclosure;

FIG. 2 shows the circulatory assist device of FIG. 1 in a deployed (e.g., expanded state) position, with sections of a pulsatory component of the circulatory assist device in an unactuated (operationally expanded state) position, in accordance with embodiments of this disclosure;

FIG. 3 shows the circulatory assist device of FIG. 1 in a deployed (e.g., expanded state) position, with the sections of the pulsatory component of the circulatory assist device in an actuated (e.g., operationally constricted state) position, in accordance with embodiments of this disclosure;

FIG. 4 is an enlarged view of a portion of the circulatory assist device including a section of the rotary component of the circulatory assist device of FIG. 3 in an actuated (e.g., operationally constricted state) position, in accordance with embodiments of this disclosure;

FIG. 5 is an enlarged cutaway view of a central portion of the driveline of the circulatory assist device of FIG. 3 illustrating the electromagnetic component associated with the second portion of the pulsatory component of the circulatory assist device, in accordance with embodiments of this disclosure;

FIG. 6 shows results from a finite element analysis of simulated blood flow through a pulsatory component of the circulatory assist device of FIG. 1;

FIG. 7 shows results from a finite element analysis of simulated blood flow through a rotary component of the circulatory assist device of FIG. 1; and

FIG. 8 shows results from a finite element analysis of simulated blood flow through the circulatory assist device of FIG. 1 including both the rotary component and the pulsatory component.

### DETAILED DESCRIPTION

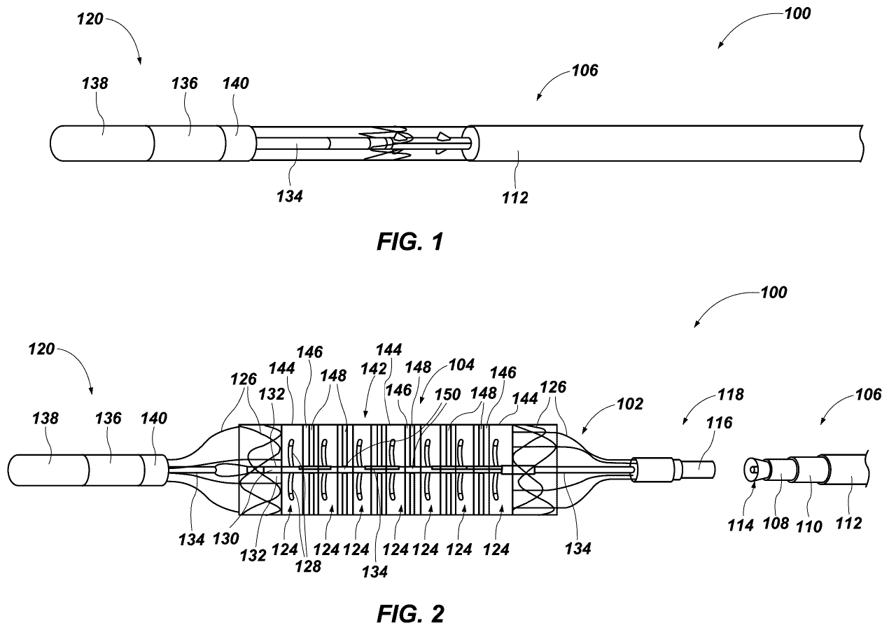

FIGS. 1-2 show a circulatory assist device 100 in a stowed (e.g., a collapsed state) position (FIG. 1) and in a deployed (e.g., an expanded state) position (FIG. 2). The circulatory assist device 100 is configured to be inserted into the blood vessel(s) of a subject (e.g., a mammal, such as a human) to facilitate pulsatile blood flow within the subject's blood vessel(s). The circulatory assist device 100 may be inserted into and positioned in a desired location within any desired blood vessel(s), such as the descending aorta above (e.g., upstream of) of the subject's renal arteries, the descending aorta below (e.g., downstream of) the subject's renal arteries, in the ascending thoracic aorta above the origin of coronary arteries and below the Innominate artery, any other peripheral artery, or any peripheral vein, the Inferior Vena Cava, or the Superior Vena Cava.

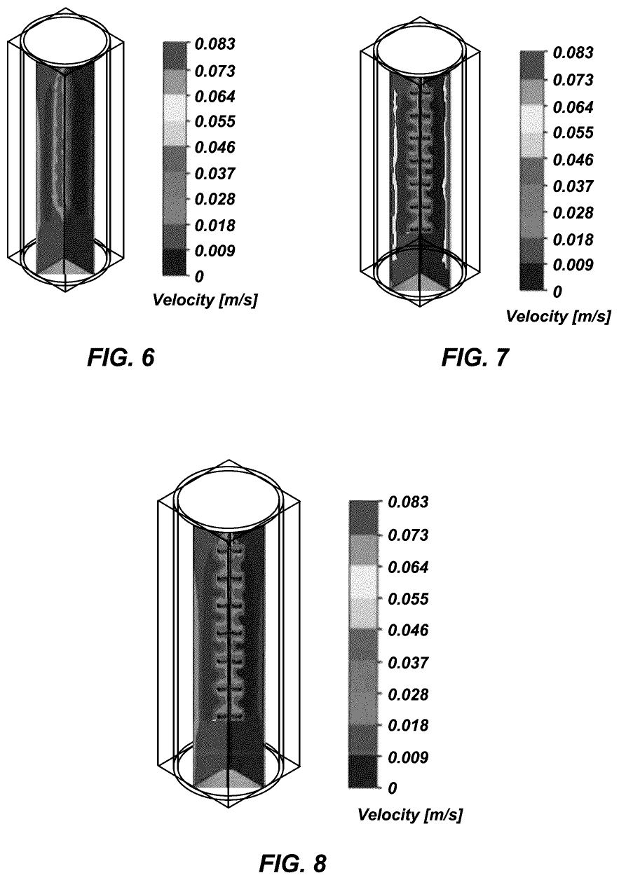

Referring collectively to FIGS. 1 and 2, the circulatory assist device 100 generally includes a rotary component 102, a pulsatory component 104, and a stent cage 126. The rotary component 102 and the pulsatory component 104 of the circulatory assist device 100 may each individually be capable of functioning independently from one another to facilitate blood flow within the subject's blood vessel(s). However, as shown and described herein (e.g., with reference to FIGS. 6-8), the rotary component 102 and pulsatory component 104 may be combined and function cooperatively to further enhance pulsatile blood circulation through the subject's blood vessel(s) relative to the individual operation of each of the rotary component 102 and the pulsatory component 104.

To facilitate introduction into and removal from the subject's blood vessel(s), the circulatory assist device 100 may, optionally, be included in a circulatory assist system that includes a catheter 106 configured to connect to the circulatory assist device 100. Referring to FIG. 2, the catheter 106 includes an inner member 108, a middle member 110, and an outer member 112, each arranged coaxially, and concentrically relative to one another. Each of the inner member 108, the middle member 110, and the outer member 112 may be configured to axially translate relative to one another (e.g., in a telescoping arrangement) to facilitate receipt of the circulatory assist device 100 within the catheter 106 for insertion and removal of the circulatory assist device 100 into and out of a blood vessel of the patient. The catheter 106 additionally includes a connection feature 114 configured to connect to a small portion 116 at a proximal end 118 (e.g., docking end) of the circulatory assist device 100. The connection feature 114 may include fingers with radially movable ends arranged circumferentially around a central pin, the fingers configured to clasp the small portion 116.

To connect the catheter 106 to the circulatory assist device 100, the catheter 106 may also include a central pin. The central pin of the catheter 106 may be received within a recess within the small portion 116 of the circulatory assist device 100. The outer member 112 may be axially advanced relative to the remainder of the catheter 106 toward the circulatory assist device 100 until the edge of the outer member 112 abuts the distal end portion at the distal end 120 (e.g., the drive end distal to the small portion 116) of the circulatory assist device 100, and all or at least the majority of the circulatory assist device 100 is encompassed within the outer member 112 of the catheter 106 (e.g., all of the components of the circulatory assist device 100 except for the distal end portion is encompassed within the outer member 112). Advancing the outer member 112 of the catheter 106 over the circulatory assist device 100 may collapse the central portion of the circulatory assist device 100 such that the outer member 112 encompasses the majority of the circulatory assist device 100. Thus, after connecting the catheter 106, the radial footprint and cross-sectional area of the circulatory assist device 100 is temporarily reduced to facilitate introduction into and/or removal from the subject's blood vessel(s).

To disconnect the catheter 106 from the circulatory assist device 100, the outer member 112 of the catheter 106 may be retracted (e.g., axially) toward the proximal end 118 of the circulatory assist device 100 and remainder of the catheter 106. The fingers may release the small portion 116 after the outer member 112 is retracted from and is no longer positioned radially outward of the circulatory assist device 100.

As shown in FIGS. 1 and 2, the circulatory assist device 100 is configured to transition from a stowed position/arrangement (e.g., collapsed state) (FIG. 1) to a deployed position/arrangement (e.g., expanded state) (FIG. 2), and vice versa. To facilitate introduction and removal into a blood vessel of a patient, the circulatory assist device 100 is arranged in the collapsed state to temporarily reduce the radial footprint/cross-sectional area of the circulatory assist device 100, which allows the circulatory assist device 100 to move within the blood vessel. As non-limiting examples, the diameter of the circulatory assist device 100 in the collapsed state may be from about 4 millimeters (mm) to about 10 mm, such as about 4 mm, about 5 mm, about 6 mm, about 7 mm, about 8 mm, about 9 mm, or about 10 mm. In the expanded state, the outer diameter of the circulatory assist device 100 may be any desired size for the blood vessel into which the circulatory assist device 100 will be deployed and operable such that the outer walls of the circulatory assist device 100 can brace against the walls of the subject's blood vessel(s) while still allowing for pulsatile movements of the blood vessel(s). As non-limiting examples, the outer diameter of the circulatory assist device 100 in the deployed state may be from about 18 mm to about 24 mm, such as about 18 mm, about 19 mm, about 20 mm, about 21 mm, about 22 mm, about 23 mm, or about 24 mm. In some embodiments, each of the stent cage 126, the pulsatory component 104, and the rotary component 102 is configured to transition from a stowed position including a radial footprint substantially small enough to fit within an outer member 112 of the catheter 106 and a deployed position including a radially expanded footprint substantially large enough for the stent cage 126 to contact an inner wall of the blood vessel.

After inserting the circulatory assist device 100 into the subject (e.g., utilizing the Seldinger technique within the subject's femoral artery) and positioning the circulatory assist device 100 in a desired location in the subject's blood vessel(s) (e.g., above the subject's renal arteries in the descending aorta), the circulatory assist device 100 may transition from the collapsed state (FIG. 1) to the expanded state (FIG. 2). After positioning the circulatory assist device 100 in the expanded state, the catheter 106 may be disconnected from the circulatory assist device 100 and withdrawn from the subject. Furthermore, the circulatory assist device 100 (e.g., the rotary component 102 and/or the pulsatory component 104) may be activated (e.g., via wireless energy or battery power) to facilitate blood circulation within the subject.

The rotary component 102 of the circulatory assist device 100 generally includes one or more impeller(s) 124 (seven shown) arranged in series. The one or more impeller(s) 124 may be encompassed within a stent cage 126, portions of the stent cage 126, within the pulsatory component 104, or a combination thereof. Each of the impellers 124 includes one or more impeller blade(s) 128 (e.g., two impeller blades 128 per impeller 124 as illustrated in FIG. 2) that can be stowed within and or deployed from one or more pocket(s) 132 formed in a casing 130. In some embodiments, each of the pockets 132 is configured to receive a respective one of the impeller blades 128 of the respective impeller 124 (e.g., the impeller 124 axially adjacent to a respective pocket 132. The impellers 124 may be connected to one another along a common driveline 134 that spans from the proximal end 118 (e.g., the docking end) to the distal end 120 (e.g., the drive end) of the circulatory assist device 100. The driveline 134 may include the casing 130 and the one or more pockets 132 formed therein. The driveline 134 may be supported by bearings at the distal end 120 and the proximal end 118 of the circulatory assist device 100 to facilitate rotation thereof. To rotate the impellers 124, the end portion of the distal end 120 may include a power supply 136 (e.g., a battery), circuitry 138, and a motor 140. The circuitry 138 may comprise a wireless charging circuit, a communications circuit, and a control circuit. The power supply 136, the circuitry 138, and the motor 140 may be housed within a canister (e.g., a hermetically sealed canister) covering and sealing the components therein.

Rotation of the impellers 124 may be controlled in several ways. For example, the impellers 124 may be driven (e.g., rotated) internally via the power supply 136, the circuitry 138, and/or the motor 140, or externally via inductive coupling (e.g., from a belt including a coil worn about the torso (e.g., the thorax) of the subject. Thus, the rotary component 102 utilizes the impellers 124 to facilitate pulsatile blood flow within the subject's blood vessel(s). Further examples of the impellers 124 include the impeller devices and methods of driving the impeller devices described in U.S. application Ser. No. 17/698,287, entitled “Circulatory Assist Pump,” to Leonhardt (Mar. 18, 2022), U.S. Pat. No. 17,470,930, entitled “Circulatory Assist Pumps, Abdominal Belts for Charging Circulatory Assist Pumps, Deployment Catheters, Retrieval Catheters, and Related Systems and Methods” to Richardson (Sep. 9, 2021), the contents of each of which are hereby incorporated herein by this reference.

The pulsatory component 104 of the circulatory assist device 100 generally includes a stent graft 142. At least a portion of the stent graft 142 may be configured to change diameter (e.g., expand or contract) in response to being actuated (e.g., by an electromagnetic field, an electric field, or a magnetic field, electric current, etc.). For example, the stent graft 142 may include one or more sections, each section including a first portion 144 and a second portion 146 (e.g., an actuatable portion). The second portion 146 is configured to constrict or expand to change diameter in response to an applied stimulus or removal of the applied stimulus. The second portion 146 may be selectively actuatable to constrict and/or expand the second portion 146 and/or at least part of the stent graft 142 to facilitate pulsatile blood flow within the subject's blood vessel(s). The first portion 144 may partially deform due to the constriction of the second portion 146.

In some embodiments, the sections of the first portion 144 and the second portion 146 are arranged end-to-end along the length of the stent graft 142. In additional embodiments, each section of the stent graft 142 may include linear sub-sections of the first portion 144 and the second portion 146 arranged adjacent to one another around the circumference of the stent graft 142. In such embodiments, the first portion 144 and the second portion 146 may extend a partial or full longitudinal length of the stent graft 142 (e.g., parallel to an axis of the stent graft 142). In further embodiments, the stent graft 142 may include an inner member and an outer member. The inner member may include the first portion 144 and the second portion 146, and the outer member may be substantially similar (e.g., in material and structure) to the first portion 144. The stent graft 142 may be substantially similar to any of those described in U.S. Patent Application Publication 2022/0117719 A1, entitled “Pulsatile Vascular Stent Graft,” to Leonhardt (Apr. 21, 2022); and U.S. Application No. 63/348,364, entitled “Pulsating Stent Graft with Implanted Flexible Electromagnetic Coil or Magnetically Activated Band Actuator to Improve Cardiac Function and Renal Blood Flow,” the contents of each of which are incorporated herein by this reference.

In some embodiments, the second portion 146 of the stent graft 142 may include one or more ferroic material(s) 148, such as ferroelectric materials (e.g., dielectric material(s), ferromagnetic materials, ferroelastic materials, or multiferroic materials. In some embodiments, the ferroic material(s) include one or more dielectric polymer(s)) and electrical leads connected to the ferroic material(s) 148 to configured to supply an electric current to the ferroic material(s) 148 to actuate the second portion 146. The ferroic material(s) 148 may be sensitive to an electric field and/or heat. Accordingly, an electromagnetic field, an electric field, and/or heat may be applied to the second portion 146, and in particular, the ferroic material(s) 148, to actuate the second portion 146.

In additional embodiments, the second portion 146 of the stent graft 142 includes one or more ferroic material(s) 148 (e.g., ferroelectric polymer) that are sensitive to an electric or electromagnetic field to actuate the second portion 146. In further embodiments, the second portion 146 of the stent graft 142 includes one or more ferroic material(s) 148 (six shown), such as elongated ferromagnetic elements, ferromagnetic particles, etc., that are sensitive to a magnetic field or electromagnetic field to actuate the second portion 146. In additional embodiments, the ferroic materials 148 may include a combination of one or more of the ferroelectric material(s) and/or one or more ferromagnetic material(s).

To actuate the second portion 146 of the stent graft 142 of the pulsatory component 104 of the circulatory assist device 100, the pulsatory component 104 may additionally include internal electromagnetic components 150 (two shown) in electronic communication with the power supply 136. The electromagnetic components 150 may include one or more electromagnets 160 (FIG. 5) that can be independently activated and deactivated to generate an electromagnetic (e.g., a magnetic field) that may attract and/or repel the ferroic material 148 within the second portion 146 of the stent graft 142. The second portion 146 of the stent graft 142 of the pulsatory component 104 may expand and/or contract in response to the attraction or repulsion of the ferroic material 148 within the second portion 146, which may facilitate pulsatile blood flow within the subject's blood vessel(s).

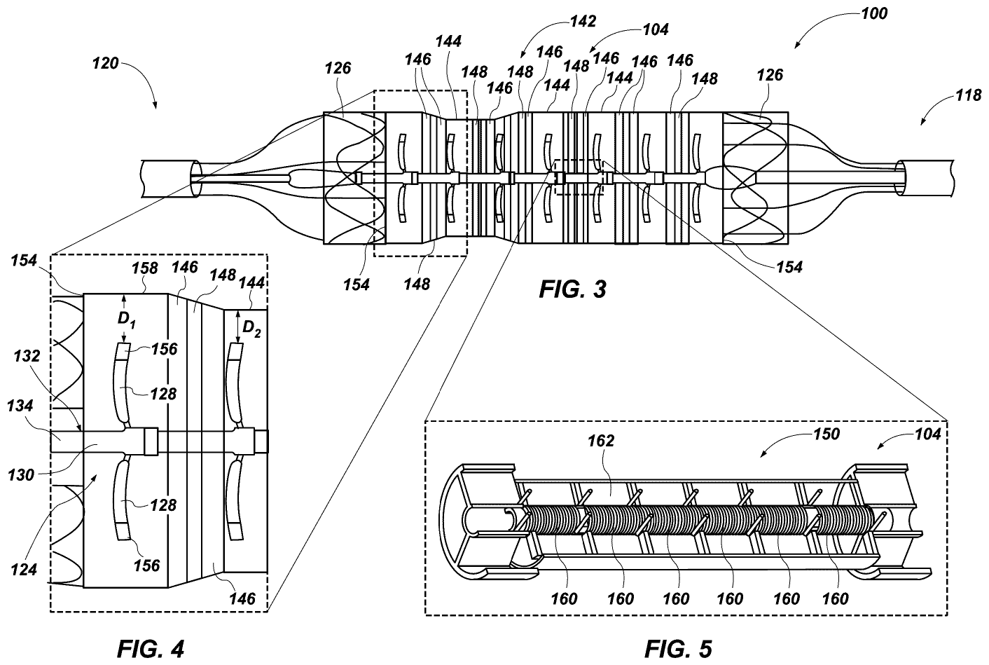

FIG. 3 shows an enlarged view of the circulatory assist device of FIG. 2 in the expanded state, with an actuatable portion of a pulsatory component 104 of the circulatory assist device 100 in an actuated (e.g., operationally constricted state) position, in accordance with embodiments of this disclosure. As shown in FIG. 3, the stent graft 142 of the pulsatory component 104 may be integrated into the stent cage 126 or may extend between portions of the stent cage 126. In some embodiments, ends 154 of the stent graft 142 may be secured to ends of the stent cage 126 such that the stent graft 142 replaces the central portion of the stent cage 126. In additional embodiments, the stent graft 142 may be radially internal relative to the stent cage 126 and/or radially external to the stent cage 126. For example, the stent graft 142 may be a first tubular member that is radially within or radially outside of a second tubular member (e.g., the stent cage 126). A first portion 144 of the sections at the ends of the stent graft 142 may be secured to portions of the stent cage 126, and the second portion 146 may be free to move (e.g., constrict radially) relative to the stent cage 126. In additional embodiments, the second portion 146 of the sections at the ends of the stent graft 142 may be secured to the stent cage 126, actuation of which, may cause the stent cage 126 to partially deform while the stent cage 126 maintains sufficient outward radial pressure to secure the circulatory assist device 100 against the inner wall of the blood vessel to maintain a position thereof within the blood vessel.

FIG. 4 an enlarged view of actuated portion of the circulatory assist device including a section of the pulsatory component 104 of the circulatory assist device 100 of FIG. 3 in an actuated (e.g., operationally constricted state) position, in accordance with embodiments of this disclosure. In FIG. 4, the impeller blades 128 are illustrated in the deployed position. In the deployed position, the tips 156 of the impeller blades 128 may be oriented substantially perpendicularly to the casing 130 and/or the driveline 134. In addition, because the stent graft 142 of the pulsatory component 104 may be configured to constrict in response to actuation of the second portion 146, the length of the impeller blades 128 may be selected such that a distance D1 from the blade tips 156 to the interior wall 158 of the stent graft 142 is sufficient to accommodate actuation and maximum contraction of the second portion 146 of the stent graft 142 (e.g., the maximum extent of the operationally constricted state) without contacting the rotating impeller blades 128. At least a portion (e.g., the second portion 146) of the stent graft 142 may be configured to constrict from about 1 mm to about 10 mm, such as about 1 mm, about 2 mm, about 3 mm, about 4 mm, about 5 mm, about 6 mm, about 7 mm, about 8 mm, about 9 mm, or about 10 mm. Accordingly, the distance D1 (which is a radial measurement) may be slightly larger than half of the distance the stent graft 142 is configured to move during actuation. Thus, if the maximum movement (e.g., constriction) of the portion of the stent graft is X, the distance D1 is at least X/2, and may be any number larger than X/2, such as X/2+1, X/2+2, etc. For example, in embodiments in which at least a portion (e.g., the second portion 146) of the stent graft 142 constricts 6 mm from the fully expanded state, the distance D1 may be at least 3 mm, such as about 4 mm, 5 mm, or about 6 mm. Thus, the length of the impeller blades 128 may be reduced to accommodate constricting movement of the stent graft 142.

FIG. 5 is an enlarged cutaway view of a portion of the driveline 134 of the circulatory assist device 100 of FIG. 3, illustrating the electromagnetic components 150 associated with the second portion 146 of the pulsatory component 104 of the circulatory assist device 100, in accordance with embodiments of this disclosure. As shown in FIG. 5, the electromagnetic components 150 includes one or more electromagnets 160 (six shown) arranged in series along a length of a casing 162. The casing 162 may be transparent to electromagnetic waves to facilitate functioning of the device. The electromagnets 160 may be independently actuated. Accordingly, each of the individual electromagnets 160 may be independently (e.g., sequentially) activated to facilitate directional actuation of the second portion 146 of subsequent sections of the stent graft 142 to facilitate directional blood flow. For example, all of the electromagnets 160 within each of the electromagnetic components 150 may be activated (e.g., sequentially), pairs of the electromagnets 160 within the electromagnetic components 150 may be independently (e.g., sequentially) activated. Each of the electromagnets 160 may be positioned and configured to actuate a respective second portion 146, and in particular, a respective ferroic material 148.

FIG. 6 shows results from a finite element analysis of simulated blood flow through the pulsatory component 104 of the circulatory assist device 100 that is expanded and in operation, and which is isolated from the rotary component 102 of the circulatory assist device 100. From the results shown in FIG. 6, the peak velocity of blood flow is achieved roughly equidistantly between the inner walls and the center of the pulsatory component 104, with areas of low flow velocity occurring near the center of the pulsatory component 104.

FIG. 7 shows results from a finite element analysis of simulated blood flow through the rotary component 102 of the circulatory assist device 100 that is expanded and in operation, and which is isolated from the pulsatory component 104 of the circulatory assist device 100. From the results shown in FIG. 7, the peak velocity of blood flow is proximate to the center of the pulsatory component 104 near the impellers 124, with areas of low flow velocity occurring near the exterior walls of the pulsatory component 104.

FIG. 8 shows results from a finite element analysis of simulated blood flow through the circulatory assist device 100 that is expanded and in operation, utilizing both the rotary component 102 and the pulsatory component 104 of the circulatory assist device 100. From the results shown in FIG. 8, the areas of low flow velocity are mitigated when the combination of the rotary component 102 and the pulsatory component 104 cooperatively act to facilitate blood flow within the subject's blood vessel(s).

Once being apprised of the instant disclosure, one of ordinary skill in the art would be readily able to make the described circulatory assist device.

In the application above, the claims below, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

The following description provides specific details, such as components, assembly, and materials in order to provide a thorough description of embodiments of the disclosure. However, a person of ordinary skill in the art will understand that the embodiments of the disclosure may be practiced without employing these specific details.

The use of the term “for example,” means that the related description is explanatory, and though the scope of the disclosure is intended to encompass the examples and legal equivalents, the use of such terms is not intended to limit the scope of an embodiment or this disclosure to the specified components, acts, features, functions, or the like.

Drawings presented herein are for illustrative purposes only, and are not meant to be actual views of any particular material, component, structure, or device. Thus, embodiments described herein are not to be construed as being limited to the particular shapes or regions as illustrated, but include deviations in shapes that result, for example, from manufacturing. For example, a region illustrated or described as box-shaped may have rough and/or nonlinear features, and a region illustrated or described as round may include some rough and/or linear features. Moreover, sharp angles that are illustrated may be rounded, and vice versa. Thus, the regions illustrated in the figures are schematic in nature, and their shapes are not intended to illustrate the precise shape of a region and do not limit the scope of the present claims. The drawings are not necessarily to scale. Additionally, elements common between figures may retain the same numerical designation.

As used herein, the term “configured” refers to a size, shape, material composition, material distribution, orientation, and arrangement of one or more of at least one structure and at least one apparatus facilitating operation of one or more of the structure and the apparatus in a predetermined way.

As used herein, the terms “comprising” and “including,” and grammatical equivalents thereof include both open-ended terms that do not exclude additional, unrecited elements or method acts, and more restrictive terms such as “consisting of” and “consisting essentially of” and grammatical equivalents thereof.

As used herein, the term “may” with respect to a material, structure, feature, or method act indicates that such is contemplated for use in implementation of an embodiment of the disclosure and such term is used in preference to the more restrictive term “is” so as to avoid any implication that other, compatible materials, structures, features and methods usable in combination therewith should or must be excluded.

As used herein, the singular forms “a,” “an,” and “the” include the plural forms as well, unless the context clearly indicates otherwise.

As used herein, the term “and/or” includes any and all combinations of one or more of the associated listed items.

As used herein, relational terms, such as “first,” “second,” etc., are used for clarity and convenience in understanding the disclosure and accompanying drawings and does not connote or depend on any specific preference, orientation, or order, except where the context clearly indicates otherwise.

As used herein, the term “about,” when used in reference to a numerical value for a particular parameter, is inclusive of the numerical value and a degree of variance from the numerical value that one of ordinary skill in the art would understand is within acceptable tolerances for the particular parameter. For example, “about,” in reference to a numerical value, may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

As used herein, the terms “biocompatible material” and “biocompatible materials” refer to any materials suitable for being within a subject's body. Biocompatible materials include ceramics and ceramic composites such as alumina (Al₂O₃), zirconia (ZrO₂), hydroxyapatite (Ca₁₀(PO₄)₆(OH)₂), and bioglass (e.g., composites including silica (SiO₂), calcium (Ca), sodium oxide (Na₂O), hydrogen (H), and/or phosphorous (P)). As non-limiting examples, bioglass may include 45S5 (e.g., 45% SiO₂, 24.5% CaO, 24.5% Na₂O, and 6% (P₂O₅) and additional compositions described in the following article, the contents of which are incorporated herein by this reference: Vidya Krishnan and T. Lakshmi, “Bioglass: A novel biocompatible innovation,” Journal of Advanced Pharmaceutical Technology & Research, Volume 4(2), April-June 2013, pages 78-83, DOI: 10.4103/2231-4040.111523. Biocompatible materials may additionally include metals and metal alloys, such as stainless steel, titanium and titanium alloys (e.g., Nitinol), cobalt-chromium alloys (e.g., ASTM F75). Furthermore, biocompatible material may include polymers, such as polyvinylchloride (PVC), polyethylene (PE), polypropylene (PP), polytetrafluoroethylene (PTFE), expanded PTFE (ePTFE), polyetheretherketone (“PEEK”), Poly-paraphenylene terephthalamide (K29) (e.g., Kevlar®), p-phenylene terephthalamide (PpPTA) (e.g., Twaron®), polymethylmethacrylate (PMMA), trimethylcarbonate (C₄H₆O₃), TMC NAD-lactide (CH₃[C₆H₈O₄]ₘ[C₄H₆O₃]ₙCH₃), polylactic acid (PLA), and medical-grade silicone.

### REFERENCES

(the contents of each of which are incorporated herein by this reference)

PCT International Patent Publication WO2019183247A1, entitled “Circulatory assist pump” to Leonhardt (Sep. 26, 2019).

U.S. application Ser. No. 17/698,287, entitled “Circulatory Assist Pump” to Leonhardt (Mar. 18, 2022).

U.S. Pat. No. 17,470,930, entitled “Circulatory Assist Pumps, Abdominal Belts for Charging Circulatory Assist Pumps, Deployment Catheters, Retrieval Catheters, and Related Systems and Methods” to Richardson (Sep. 9, 2021).

U.S. Patent Application Publication 2022/0117719 A1, entitled “Pulsatile Vascular Stent Graft,” to Leonhardt (Apr. 21, 2022).

U.S. Application No. 63/348,364, entitled “Pulsating Stent Graft with Implanted Flexible Electromagnetic Coil or Magnetically Activated Band Actuator to Improve Cardiac Function and Renal Blood Flow” filed Jun. 2, 2022.

1. A circulatory assist device comprising:
a stent cage;
a pulsatory component configured to facilitate pulsatile blood flow through a blood vessel of a subject, the pulsatory component comprising a stent graft integrated in at least a portion of the stent cage, the stent graft including one or more sections configured to diametrically constrict in response to an applied stimulus; and
a rotary component configured to facilitate the pulsatile blood flow through the blood vessel of the subject, the rotary component comprising an impeller encompassed by at least a portion of the stent graft, the impeller and the one or more sections are configured to act cooperatively to facilitate blood flow within the blood vessel of the subject,
wherein each of the one or more sections includes a ferroic material configured to cause a respective section of the one or more sections to constrict in response to the applied stimulus.

2. (canceled)

3. The circulatory assist device of claim 1, wherein the impeller includes a driveline and one or more impeller blades extending from the driveline.

4. The circulatory assist device of claim 3, further comprising at least one electromagnet positioned within the driveline, and wherein the at least one electromagnet is configured to cause the applied stimulus.

5. The circulatory assist device of claim 4, further comprising circuitry configured to cause constriction and expansion of the one or more sections by controlling the at least one electromagnet and emission of the applied stimulus thereby.

6. The circulatory assist device of claim 5, further comprising a motor coupled to the driveline, and wherein the circuitry is configured to control rotation of the impeller via the motor simultaneously with the constriction and the expansion of the one or more sections.

7. The circulatory assist device of claim 1, wherein each of the stent cage, the pulsatory component, and the rotary component is configured to transition from a stowed position including a radial footprint substantially small enough to fit within an outer casing of a catheter and a deployed position including a radially expanded footprint substantially large enough for the stent cage to contact an inner wall of the blood vessel.

8. A circulatory assist device for facilitating pulsatile blood flow within a subject's blood vessel, the circulatory assist device comprising:
a rotary component comprising a driveline and one or more impellers connected to the driveline and configured to rotate with the driveline;
a pulsatory component comprising one or more sections configured to change diameter in response to a magnetic field applied thereto; and
one or more electromagnets positioned within the driveline and configured to produce the magnetic field.

9. The circulatory assist device of claim 8, wherein each of the one or more sections includes a ferroic material configured to constrict in response to the magnetic field being applied thereto.

10. The circulatory assist device of claim 8, wherein each of the pulsatory component and the rotary component is configured to transition from a stowed position including a radial footprint substantially small enough to fit within an outer casing of a catheter and a deployed position including a radially expanded footprint larger than the radial footprint.

11. The circulatory assist device of claim 8, further comprising circuitry configured to cause constriction and expansion of the one or more sections by controlling the one or more electromagnets and production of the magnetic field thereby.

12. The circulatory assist device of claim 11, further comprising a motor coupled to the driveline, and wherein the circuitry is configured to control rotation of the one or more impellers via the motor simultaneously with the constriction and the expansion of the one or more sections.

13. The circulatory assist device of claim 8, further comprising a stent cage positioned on each side of the pulsatory component, the stent cage on each side of the pulsatory component configured to support the pulsatory component.

14. The circulatory assist device of claim 9, wherein the ferroic material includes one or more material selected from among ferroelectric material and ferromagnetic material.

15. A method for facilitating pulsatile blood flow within a blood vessel of a subject, the method comprising:
introducing a circulatory assist device into the blood vessel, the circulatory assist device including:
a pulsatory component configured to facilitate pulsatile blood flow through a blood vessel of a subject, the pulsatory component comprising a stent graft integrated in at least a portion of a stent cage, the stent graft including one or more sections configured to diametrically constrict in response to an applied stimulus; and
a rotary component configured to facilitate the pulsatile blood flow through the blood vessel of the subject, the rotary component comprising an impeller encompassed by at least a portion of the stent graft, the impeller and the one or more sections are configured to act cooperatively to facilitate blood flow within the blood vessel of the subject,
wherein each of the one or more sections includes a ferroic material configured to cause a respective section of the one or more sections to constrict in response to the applied stimulus;
causing a driveline of the rotary component to rotate the impeller; and
causing at least one of the one or more sections of the pulsatory component to change diameter by applying a magnetic field thereto using one or more electromagnets positioned within the driveline, the one or more electromagnets configured to produce the magnetic field.

16. The method according to claim 15, further comprising causing each of the pulsatory component and the rotary component to transition from a stowed position including a radial footprint substantially small enough to fit within an outer casing of a catheter to a deployed position including a radially expanded footprint larger than the radial footprint after introducing the circulatory assist device into the blood vessel.

17. The method according to claim 16, wherein the stent cage is positioned at least on each side of the pulsatory component and the radially expanded footprint is substantially large enough for the stent cage to contact an inner wall of the blood vessel.

18. The method according to claim 16, further comprising, prior to removing the circulatory assist device from the blood vessel causing each of the pulsatory component and the rotary component to transition to the stowed position from the deployed position including causing one or more impeller blades of the impeller to be stowed within pockets formed by a casing of the driveline.

19. The method according to claim 15, wherein causing the driveline of the rotary component to rotate the impeller and causing the at least one of the one or more sections of the pulsatory component to change diameter by applying the magnetic field thereto are performed simultaneously.

20. (canceled)
